(19) United States
(12) Patent Application Publication
(10) Pub. No.: US 2025/0090087 A1
(43) Pub. Date: Mar. 20, 2025

(54) SYSTEMS AND METHODS FOR ENHANCING DIALOGUE INTERACTION SKILLS

(71) Applicant: HOFSTRA UNIVERSITY, Hempstead, NY (US)

(72) Inventors: Patrizia Bonaventura, Locust Valley, NY (US); Krishnan Pillaipakkamnatt, Westbury, NY (US)

(21) Appl. No.: 18/886,665

(22) Filed: Sep. 16, 2024

Related U.S. Application Data
(60) Provisional application No. 63/538,326, filed on Sep. 14, 2023.

(51) Int. Cl. A61B 5/00; B25J 11/00

(52) U.S. Cl. CPC A61B 5/4803; A61B 5/741; B25J 11/0005

(57) ABSTRACT

In accordance with aspects of the disclosure, a system for training conversation skills, dialogue interaction skills, and pragmatic language skills in a patient with autism spectrum disorder (ASD) is presented. The system includes a robot having a non-humanoid, non-animal-like shape, a processor, and a memory coupled to the processor and including instructions stored thereon. The instructions, when executed by the processor, cause the system to record an audible and/or a visual expression of the patient in proximity of the robot, record speech by a therapist and the patient, provide a predicted sentence based on the recorded at least one of audible or visual expression or the recorded speech, and audibly generate the predicted sentence. The predicted sentence is pronounced by the robot in order to elicit a verbal response from and maintain a conversation with the patient.

### CROSS-REFERENCE TO RELATED APPLICATION AND CLAIM OF PRIORITY

This application claims the benefit of, and priority to, U.S. Provisional Patent Application No. 63/538,326, filed on Sep. 14, 2023, the entire contents of which, including all appendices therein, are hereby incorporated herein by reference.

### TECHNICAL FIELD

The present disclosure relates to systems and methods for enhancing dialogue interaction skills, and, more particularly, to systems and methods for enhancing dialogue interaction skills in children with autism.

### BACKGROUND

Children with autism spectrum disorder (ASD) frequently experience neurodevelopmental dysfunctions that affect their communication skills, social interactions, and ability to develop, maintain, and understand social relationships. Social communication deficits include impairments in aspects of joint attention and social reciprocity, as well as challenges in the use of verbal and nonverbal communicative behaviors for social interaction. Furthermore, children with ASD typically exhibit restricted, repetitive behavior apparent in stereotyped motor movements, insistence on or inflexible adherence to routines, abnormal intensity when fixated on interests, and hyper- or hypo-reactivity to sensory input. Autism symptoms in children have been increasingly diagnosed, and the prevalence of autism in all regions of the world is high; 1 in 68 children is diagnosed with ASDs, according to the U.S. Centers for Disease Control and Prevention (CDC), occurring in all racial, ethnic, and socioeconomic groups. Furthermore, ASDs are almost five times more frequent among boys (1 in 42) than among girls (1 in 189).

In terms of use of language and speech, children with ASD often experience difficulties in discourse and in conversation; in particular, they show a delay in, or total lack of, the development of spoken language (without compensation, for example, through gesture or mime). In individuals with adequate speech, a marked impairment is the ability to initiate or sustain a conversation with others in maintaining the dialogue by appropriate turn taking, and in selection of appropriate topics to continue the conversation, as well as in persevering on topics based on their own interest; presence of stereotyped and repetitive use of language. Difficulties in communication, social, verbal, and motor skills are more significant, but other symptoms might co-occur, like sleep disorders, or gastrointestinal problems, as well as attention deficit hyperactivity disorder (ADHD) or intellectual disabilities.

From a linguistic point of view, children with autism show impaired pragmatic language skills and have difficulties in using speech and language in communication. Pragmatic language skills generally consist of knowing and using rules for normal verbal interaction with interlocutors; for example, establishing and maintaining eye contact during a conversation, smiling while talking, engaging others in a dialogue, maintaining comfortable speaking distances, taking conversational turns, changing topics, clarifying messages, and adding verbal or nonverbal information. Children learn these unspoken rules of language interaction, during normal language development, starting soon after birth; research suggests they may be closely linked to play skill development. Some difficulties might be normal during acquisition of these rules, as in other areas of development, but if problems persist in the use of pragmatic language skills, and interfere with the child's normal conversations and language use with her/his peers and family, then it is possible that the child develops a pragmatic disorder, as manifested in autism.

Children with pragmatic language disorders have difficulties in verbal social interaction because they might not initiate conversation, may not take turns appropriately, may talk over another speaker, or respond with inappropriate silences. A child with pragmatic language delays may interrupt excessively, shift topics abruptly, or talk irrelevantly. They may assume that every listener has knowledge of the same people and events that they do. Children with pragmatic language delays may not be aware of subtle cues people use to signal interest or discomfort. Their behavior may appear rude, distracted or self-involved. These children are known to have difficulty communicating with adults and peers, but they often demonstrate an affinity for technology, such as social robots. When interacting with social robots, children with ASD demonstrate prompted and spontaneous social behaviors, including joint attention, eye contact, understanding facial expressions and triadic interactions.

Prosody production deficits are one of the most common clinical features of ASD. Also, prosodic production differences are among the earliest characteristics of ASD, and are present at all levels of ability, including Asperger syndrome and high-functioning autism. However, these prosodic abnormalities, especially those in the production of intonation (the “melody of speech”), are not clearly defined and classified in their features. Therefore, there exists a need for a better characterization of these prosodic differences, especially in terms of intonation patterns, and to create assessment and therapy tools for prosodic disabilities (dysprosody) in children with ASD.

However, while social robots can be a powerful therapeutic tool for children with ASD who display an attraction towards technology and show “systemizing” skills, where they can recognize repeated patterns in stimuli, no non-humanoid, non-animal-like robots are known to exist that are capable of eliciting or otherwise training conversation and dialogue interaction skills as well as pragmatic language skills in children with autism.

### SUMMARY

In accordance with aspects of the disclosure, a system for training conversation skills, dialogue interaction skills, and pragmatic language skills in a patient with autism spectrum disorder (ASD) is presented. The system includes a robot having a non-humanoid, non-animal-like shape, a processor, and a memory coupled to the processor and including instructions stored thereon. The instructions, when executed by the processor, cause the system to record an audible and/or a visual expression of the patient in proximity of the robot, record speech by a therapist and the patient, provide a predicted sentence based on the recorded at least one of audible or visual expression or the recorded speech, and audibly generate the predicted sentence. The predicted sentence is pronounced by the robot in order to elicit a verbal response from and maintain a conversation with the patient.

In an aspect of the present disclosure, the audible and/or visual expression may include an emotion, a belief, and/or a communicative intention.

In another aspect of the present disclosure, the audible and/or visual expression may include a neutral, inexpressive, and/or normal tone.

In yet another aspect of the present disclosure, the recorded speech may include a pre-recorded sentence produced by a neurotypical child.

In a further aspect of the present disclosure, the recorded speech may include a sentence produced by a speech synthesizer.

In yet a further aspect of the present disclosure, the speech synthesizer may include a large language model (LLM) configured to simulate a conversation with the patient.

In an aspect of the present disclosure, the pre-recorded sentence may be audibly generated by the robot using a command on a remote control device.

In another aspect of the present disclosure, the remote control device may be a wireless remote control device.

In yet another aspect of the present disclosure, the remote control device may be a wired remote control device.

In a further aspect of the present disclosure, the predicted sentence may be configured to initiate a piloted dialogue with the patient.

In an aspect of the present disclosure, a computer-implemented method for conversation skills, dialogue interaction skills, and pragmatic language skills in a patient with autism spectrum disorder (ASD) is presented. The method includes recording an audible and/or a visual expression of the patient in proximity of a robot, recording speech by a therapist and the patient, providing a predicted sentence based on the recorded audible and/or visual expression or the recorded speech, and audibly generating the predicted sentence. The predicted sentence is pronounced by the robot in order to elicit a verbal response from and maintain a conversation with the patient.

Further details and aspects of the present disclosure are described in more detail below with reference to the appended figures.

### BRIEF DESCRIPTION OF THE DRAWINGS

A better understanding of the features and advantages of the present disclosure will be obtained by reference to the following detailed description that sets forth illustrative aspects, in which the principles of the present disclosure are utilized, and the accompanying figures of which:

FIG. 1 is an exemplary therapy system 100, according to one or more embodiments of the present disclosure;

FIG. 2 is an exemplary robot configurable for implementing a therapy method for training and improving conversation and dialogue interaction skills as well as pragmatic language skills in children with autism, according to one or more embodiments of the present disclosure;

FIGS. 3A and 3B are an exemplary robot configurable for implementing a therapy method for training and improving conversation and dialogue interaction skills as well as pragmatic language skills in children with autism, according to one or more embodiments of the present disclosure; and

FIG. 4 is a diagram conceptually depicting robot classification based on shape, ranked in order of most verbal responses elicited by the robot (top row) to least number of verbal responses (bottom row).

### DETAILED DESCRIPTION

Principles of the present disclosure will be described herein in the context of an illustrative apparatus, system and therapy method for training and improving conversation and dialogue interaction skills as well as pragmatic language skills in children with autism spectrum disorder (ASD). Specifically, the disclosure provides a tool, which can record the dialogues between children with ASD and a robot, thereby allowing analysis and identification of patterns of dysprosody in intonation in the children's speech and improved diagnosis and/or treatment of ASD.

Connected with the problem of the nature and causes of the dysprosody in verbal children with ASD, is the problem of the language spoken by the children and whether the deficits in pitch, loudness and intonation would be typical of each language, or common to all children with ASD across languages and cultures. Prosodic patterns are similar in ASD children for at least three languages (English, Italian and Tamil), indicating that the type of dysprosody might be a symptom of ASD independent from the prosody of the language the children with ASD speak. Therefore, the robot is configured to speak sentences in at least the three languages, Italian, English and Tamil, and has been tested in different countries, providing good results in all the experimental conditions. Moreover, dialogues in different languages can be added simply, e.g., via an ad hoc app, to trigger production of the sentences recorded in different languages.

Furthermore, by using the app to control the robot's speech, different models of piloted dialogues can be experimented with, including different features in terms of semantic content (general topics and meanings of the sentences), of emotional content and of communication intentions (all these three levels of expression are related and conveyed by the intonation of speech): the manipulation of the intonation contours both in natural speech and in synthesized speech, can provide the necessary association between the appropriate response in dialogue interaction in terms of linguistic features (e.g. appropriate topic, adequate turns, comprehension of intentions of the speaker in the conversation) and the right prosodic features (like emphasis on the topic of the utterance and emotions expression in intonation). Thus, the use of the robot together with different approaches of autism therapy (e.g., an ABA, or the DERBBI approach) provides the benefit of improved use of language in interaction, and the ability to sustain a conversational exchange with appropriate linguistic pragmatic skills and intonation. In aspects, the intonation in the sentences spoken by the robot can be artificially adapted to the intonation used by the child, so allowing the robot to tune to the discourse level of the child, and to teach her/him how to produce the correct intonation in dialogue.

The present disclosure provides a unique robot-based therapy approach for training and improving conversation and dialogue interaction skills as well as pragmatic language skills in children with autism. In one or more embodiments, a non-humanoid, non-animal-like robot is used as a behavioral therapy tool to speak with a child with ASD. In some embodiments, the robot is configured to move and approach the child, and to draw the child's attention by pronouncing some friendly questions. The robot is intended to speak with the voice of a real boy, pronouncing the questions and sentences to address the child with ASD. The intonation of the questions is calibrated to obtain the maximum responsiveness by the children with ASD: in particular, the sentences will be produced with two different intonations, a neutral one pronounced by a neurotypical boy and others with different emotional expressions, pronounced by a neurotypical boy. Other intonations obtained by a speech synthesizer will also be used for different tests, to verify which of the speech melodies the autistic children respond better to.

The non-humanoid non-animal-like robot is used to speak with children with ASD. Sentences pronounced by the robot aim to elicit a verbal response from the child, and to start a conversation. In this manner, the robot is designed to train and improve verbal interaction and to enhance linguistic pragmatic skills used in interpersonal communication by spoken language in autistic children. It is to be appreciated, however, that the disclosure is not limited to the specific apparatus, system and/or methods illustratively shown and described herein. Rather, it will become apparent to those skilled in the art given the teachings herein that numerous modifications can be made to the embodiments shown that are within the scope of the claimed disclosure. That is, no limitations with respect to the embodiments shown and described herein are intended or should be inferred.

After the interaction with the robot, the child with ASD will talk with an age-matched neurotypical peer, asking the same questions as the robot. We will thus compare directly the response of the children with ASD to the robot with the analogous responses of the children with ASD to the neurotypical child. Also, neurotypical children will perform the same tasks as the autistic children with the robots, and their performances will be compared to those of the autistic children, as a control group.

The sentences pronounced by the robot aim to elicit a verbal response from the child, and to thereby start and maintain a conversation. In this manner, the robot is designed to improve verbal interaction in a dialogue and to enhance linguistic pragmatic skills used in interpersonal communication by spoken language in autistic children. Linguistic pragmatic skills comprise a body of knowledge relating to interactions in conversations; for example, knowing when to take turns, selecting an appropriate topic and staying on topic, or using an appropriate tone of voice in the dialogue, and sharing the correct expression of affect, among other conversational elements.

As may be used herein, “facilitating” an action includes performing the action, making the action easier, helping to carry the action out, or causing the action to be performed. Thus, by way of example and not limitation, instructions executing on one processor might facilitate an action carried out by instructions executing on a remote processor, by sending appropriate data or commands to cause or aid the action to be performed. For the avoidance of doubt, where an actor facilitates an action by other than performing the action, the action is nevertheless performed by some entity or combination of entities.

Techniques according to embodiments of the present disclosure can provide substantial beneficial therapeutic effects. By way of example only and without limitation, one or more embodiments of the disclosure provide techniques to train conversation and dialogue interaction skills as well as pragmatic language skills in children with ASD, the embodiments having one or more of the following advantages, among other important benefits: provides a non-humanoid, non-animal-like robot configured for use in training and improving conversation and dialogue interaction skills as well as pragmatic language skills in children with ASD, as part of a unique therapy approach; ability to engage in short directed conversations with children with autism, to thereby improve the child's ability to initiate and maintain dialogue with an interlocutor; ability to trigger recognition in children with autism that there is an interlocutor present and trigger verbal interaction of the child with the conversation partner (e.g., eliciting appropriate content as a topic of the dialogue, and training appropriate pragmatic language skills to use in the conversation); creates directed dialogue through a prescribed path of interaction; uses real human voices (i.e., natural and not “electronic-sounding”) so that behaviors learned during therapy might be transferred to actual human conversational interactions.

There have been various studies conducted using different types of robot shapes ranging from humanoid to non-humanoid form and their application to improvement of communication skills for children with ASD. Ample consideration has been given to robot appearance and function in ASD research. Some robots are humanoids, while others are mechanical and non-humanoid, and still others are animal-like.

Humanoid robots resemble humans but remain repetitive and predictable in character. Using humanoid robots in interaction with children with ASD is beneficial because there may be a potential for generalization, meaning the child will use the behavior learned in research or clinical settings and transfer that learned behavior to her/his daily life. For this reason, humanoid robots can aid in behavioral imitation exercises in targeted ASD therapy.

However, robots having a humanoid shape may be less engaging to children with autism compared to non-humanoid robots. Children with ASD often withdraw themselves from human interactions and gravitate towards simple, repeatable, mechanical objects. In addition, many children with ASD can experience sensory overstimulation when exposed to an abundance of social cues: in humanoid robots, there can be a greater degree of confusing or distracting stimuli compared to other robot shapes.

Animal-like robots are modeled after animals and appear social without risking sensory overstimulation in children with ASD. These robots often are designed to train much more simplistic social cues compared to those of humanoid robots while being easier to interpret because they often allow for the expression of social cues that are simpler to decipher than those from humanoid robots. However, animal-like robots cannot simulate human-human interactions.

Non-humanoid non-animal-like robots do not resemble either human or animal form and character. Using non-humanoid non-animal-like robots in ASD research is beneficial because they can be built to complete specific tasks, are simplistic in design, and facilitate engaging interactions between child and robot. Children with ASD often withdraw themselves from human interactions; robots in a toy form often do not prompt this behavior, which can allow for greater engagement than with humanoid robots. Like that of animal-like robots, non-humanoid non-animal-like robots cannot copy human-human interactions.

The challenge in designing a robot for ASD therapy, therefore, is finding a design that engages the child without overstimulating them. To create captivating but visually simplistic robots, a cartoon-like style may be adopted in robot design, where oversized primary features, such as the eyes, may be emphasized. Robots with cartoon-like faces and machine-like bodies may provide simple, engaging stimuli for children with ASD.

Robot mobility also plays an important factor in child engagement in an ASD therapy setting and can be used as a tool to achieve varying levels of organic motion. A robot with a greater number of degrees of freedom (DOF) in the head can appear more human-like than a robot with a stationary head. All robots in ASD therapy research have some movement capability, but typically lack the ability to roll or walk around their environment. A mobile robot can provide more variety in robot-human interaction because there is a greater number of activities a robot and child can achieve together. However, robot mobility is another factor in robot design, that can also increase the cost of development.

It is important to note that robot design does not just encompass its appearance, but the clinical applications greatly influence their cosmetic and technical design. For example, if a child is set to improve their eye contact in a prescribed therapy session, a robot may be configured to have larger eyes or a camera in their eyes to track eye movement and maximize child engagement. The targeted behavior in ASD robot-assisted therapy will be factors affecting the robot's appearance and mobility in order to achieve a specific therapeutic goal.

In one or more embodiments of the disclosure, the shape of the robot is non-humanoid or non-animal-like and adapted to capture the attention of a child having ASD and to facilitate the initiation of interaction. One objective is to improve and increase the quality and complexity of spoken language exchanges in conversation.

In researching clinical studies involving the use of social robots for assessing behavior of children with ASD, participants' characteristics, study method, and study results were identified. Participant age, sex, diagnosis, and group size were reported. Robot name, study design, and skills tested of some studies are discussed in detail below. Three variables were coded in each study: (i) Does the robot have a speech function; (ii) Is there a verbal speech response to the robot in a piloted dialogue; and (iii) Was the study used for targeted speech therapy in children with ASD.

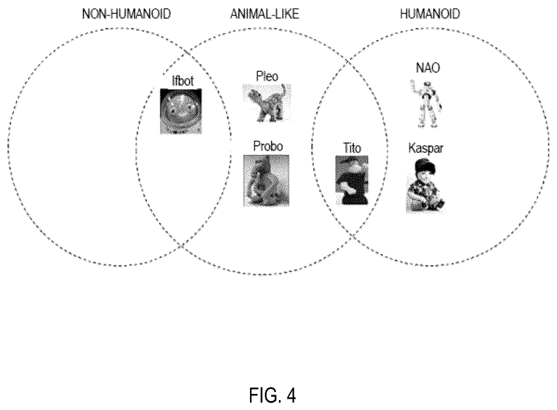

As discussed further herein, robots may be used as tools to improve the communication skills in children with ASD. Robot shapes may be ranked in decreasing order of eliciting the greatest number of verbal responses from a child with ASD to the least number of verbal responses to determine which robot shape could be best used as a mediator for speech therapy for children with ASD. FIG. 4 conceptually depicts robot classification for the noted based on shape, ranked in order of most speech responses elicited by the robot (top row) to least number of responses (bottom row). Generally, children with ASD display spontaneous verbal responses in the presence of a verbal robot.

Speech responses may be elicited from children with ASD through communication with robots mentioned, indicating robots with both humanoid and animal-like shapes have the potential to be effective tools in improving communication skills in children with ASD. For example, some social behaviors, like eye gaze and imitation, may be improved. However, humanoid and/or animal-like shapes may not be as effective in eliciting spontaneous speech responses from children with ASD. In order to address at least the above-identified need in the field of therapy for children with ASD, one or more embodiments of the disclosure beneficially provides a behavior therapy methodology that incorporates a non-humanoid, non-animal-like robot configured to train conversational and dialogue skills in children with autism. Aspects of the disclosure focus on improvement of both verbal interaction in dialogue and of linguistic pragmatic skills in use of language in speech interaction.

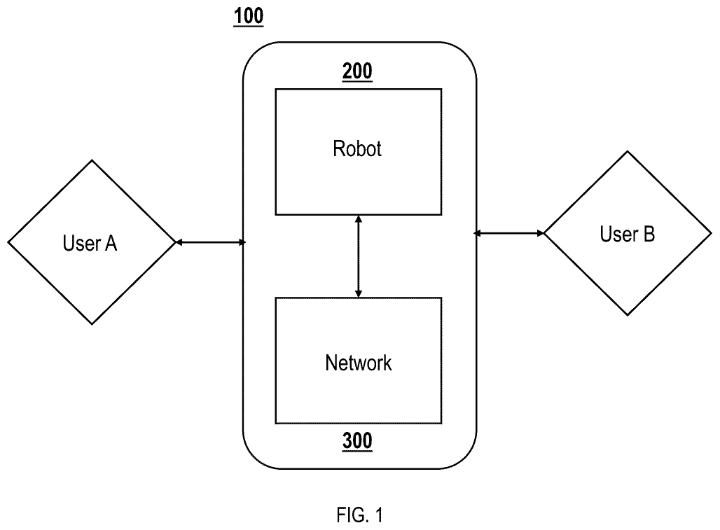

FIG. 1 shows an exemplary therapy system 100, according to one or more embodiments of the disclosure. The system 100 includes a robot 200 and a network 300. The robot is generally a non-humanoid, non-animal like robot configured to elicit sentences. The network 300 may include an external application and/or machine learning network. In use, robot 200 and network 300 may communicate to automatically generate sentences elicited to a user. For example, a user A (e.g., a therapist) may select a command which triggers network 300 to generate a sentence communicated to robot 200, which then elicits the sentence to a user B (e.g., a child patient). The following process is described further below.

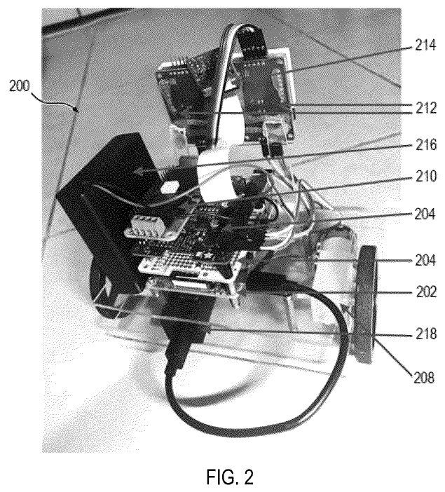

FIG. 2 depicts at least a portion of an exemplary robot 200 configurable for implementing a behavior therapy method associated with improving conversational dialogue in children with ASD, according to one or more embodiments of the disclosure. In one or more embodiments, the robot 200 is a non-humanoid non-animal-like robot configured (i.e., programmed) to speak with a child patient with ASD. Sentences pronounced by the robot are designed to elicit a verbal response from the child, and to start and maintain a conversation. In one or more embodiments, the sentences pronounced by the robot are generated based on a human being in a natural voice (e.g., sampled human voice). The sentences may be pre-recorded and programmed in the robot to be elicited automatically by a command received from a remote control device (wired or wireless). In this manner, the robot 200 seeks to improve verbal interaction in a dialogue and to enhance linguistic pragmatic skills used in interpersonal communication by spoken language in autistic children. Although a particular robot 200 is shown in FIG. 2, it is to be appreciated that embodiments of the disclosure are not limited to any specific style, dimensions and/or features of the non-humanoid robot used in conjunction with the novel behavior therapy method for improving conversational dialogue in children with ASD.

With reference to FIG. 2, the exemplary robot 200 comprises at least one processor, which in this embodiment is implemented using a Raspberry Pi 4® Model B computing module 202. The Raspberry Pi® 4 module 202 includes a processor, memory (e.g., random access memory (RAM)), and input/output (I/O) interface hardware (e.g., micro HDMI ports, gigabit Ethernet port, universal serial bus (USB) ports, etc.). The robot 200 further includes a motor driver board 204 adapted to control two or more DC steppers motors 206. The motors 206 are attached to wheels for providing movement of the robot 200.

The robot 200 includes a sound module 208, which in one or more embodiments is preferably implemented using a Raspiaudo® sound card (part of the Raspberry Pi® platform), includes two speakers and a microphone. The sound module 208 is coupled to the Raspberry Pi® computing module 202 and provides an audio interface for the robot 200. A display module 210, which in one or more embodiments is preferably implemented using a MAX7219® serially interfaced 8-digit LED display, provides a visual interface for the robot 200 for displaying expressions and other visual information generated by the computing module 202. The display module 210 is coupled to the Raspberry Pi® computing module 202 using an interface module, which is implemented in one or more embodiments using an Adafruit Perma-Proto HAT® board 212. A camera 214, which is coupled to the computing module 202, provides visual information to the processor, which can be used to monitor certain characteristics of the child, such as, for example, eye contact, facial expressions, etc., for evaluating prescribed social skills of the child.

A power module 216, which in this exemplary embodiment is implemented as a battery pack including four AA batteries, supplies power to the motor driver board 204 and DC stepper motors 206. Additionally, a portable charger 218 is provided to power the Raspberry Pi® computing module 202, the Raspiaudo® sound module 208, and the MAX7219® display module 210.

In one or more embodiments, the robot 200 comprises computer program instructions which, when executed on the processor of the computing module 202, is configured to generate sentences designed to elicit conversational dialogue with the child. The dialogues of the children with the robot are recorded in audiovisual form by the robot 200, such as by using the on-board sound module 208 and camera 214. Voice and speech acoustic parameters of the dialogue will be measured and analyzed to evaluate the progress of the child in engaging in conversational interaction with the robot 200.

More particularly, the quality of the verbal interaction in dialogue and the performance in pragmatic linguistic functions will be measured based on prescribed conversational parameters. Such prescribed conversational parameters may include, but are not limited to, one or more of the following: measuring the number of turns in the conversation; the number of appropriate turn taking; the number of initiations of dialogue; the number of topics selected by the child versus followed from the conversation; the number of appropriate topics selected by the child for the given conversation; the number of appropriate words used to express a topic; the number of different words/meanings selected; and the number of verbs and pronouns appropriately used overall and mean utterance length.

Additionally, emotional recognition and expression will be measured, in one or more embodiments, for example by evaluating the appropriateness of intonation, based on a perceptual analysis of prosodic contours. As will be known by those skilled in the art, the fundamental frequency (FO) contour in normal speech contains various types of information including emotional and other non-linguistic information such as the speaker's identity, mood and level of attention, and plays as important a role in our daily speech communication as formants, through which we encode a phonemic sequence to convey linguistic information to the listener.

Robot 200 may be configured to automatically generate and/or elicit sentences. For example, sentence production may be triggered by an application (e.g., external to the robot 200), which includes programmed instructions for sentence generation. Such sentences may be elicited in various languages, including English, Italian, and/or Tamil. In another example, a user may record speech (e.g., via an external application), which is played back through the robot 200 to a child via a command from a remote control device, thereby simulating speech by the robot 200 as if the robot was speaking to the child. In aspects, the sentences may be prerecorded, recorded live, and/or automatically generated by machine learning. For example, the robot 200 may elicit sentences based on pre-recorded sentences produced by a non-ASD child.

An intonation in the sentences produced by robot 200 may vary from a very neutral, inexpressive intonation, to a normal intonation, expressing emotions, beliefs and/or communicative intentions (e.g. questions and/or commands). In turn, an audible and/or visual expression of the child may be monitored including such intonation. Neutral tone refers to communication that is calm, balanced, impartial, and/or without strong emotional expression or bias. Inexpressive tone is characterized by a lack of emotional or vocal variation, often sounding flat, monotone, and detached, e.g., it conveys little to no enthusiasm, emotion, or energy, making the speech feel impersonal or uninterested. Normal tone refers to a balanced and natural way of speaking that reflects everyday conversation for a neurotypical individual (e.g., based on a supplied database) that typically involves moderate emotional expression and variation in pitch, volume, and pace, making the communication feel genuine and relatable.

As used herein, the “intonation” of speech refers to the means by which emotions, intentions and/or beliefs are expressed. As children with ASD have very abnormal intonation, robot 200 provides am advantage over current speech therapy by rehabilitating proper intonation, which is essential for communication. For example, by exposing the children with ASD to pronunciation of sentences with different intonations by the robot 200, the children may recognize the expressive functions of linguistic features, improving use of melody of speech in dialogue.

Robot 200 may be configured to generate a variety of sentences. For example, robot 200 may elicit a sentence asking a general question, such as: “Do you want to play with me?”; “Do you want to be my friend?” “Do you like snacks?”; “How old are you?”; or “What games do you like?” In another example, robot 200 may be configured to generate sentences that prompt a response on feelings, such as “How do you feel when you see your mom?”; “Do you see your mom often?”; “Do you like to play with the computer?”; “Are you glad when you do well in school?”; “Are you happy when you play soccer?”; or “Do you like to play with your dad?” In still another example, robot 200 may be configured to elicit sentences that prompt a response on preferences, such as: “Do you want to talk with me?”; “Would you tell me a story?”; “Would you tell me about your mom?”; “Does your mom often play with you?”; “Do you have many friends?”; “Do you like school?”; “Do you like dinosaurs?”; “What is your favorite game?”; “Do you like swimming?”; “What foods do you like?”; “Do you like puppies?”; “Do you have a puppy?”; or “Do you like cats?”.

Robot 200 may be configured to elicit sentences, which prompt a response on proper behavior and/or etiquette, such as: “What do you say if someone takes away your candy?”; “What does your mom say if you break a toy?”; “How do you feel if one of your friends gets angry with you?”; or “What do you say if you are angry with a friend?” Further, robot 200 may be configured to elicit sentences, which prompt various emotions (e.g., sad, imperious, and/or happy sentences), such as: “My mom yelled at me” (sad); “Take the snack and eat it!” (imperious); “Tomorrow I go to the beach, would you like to come with me?” (happy); “I like that car. Can I play with it?” (happy); or “Would you play with me?” (sad).

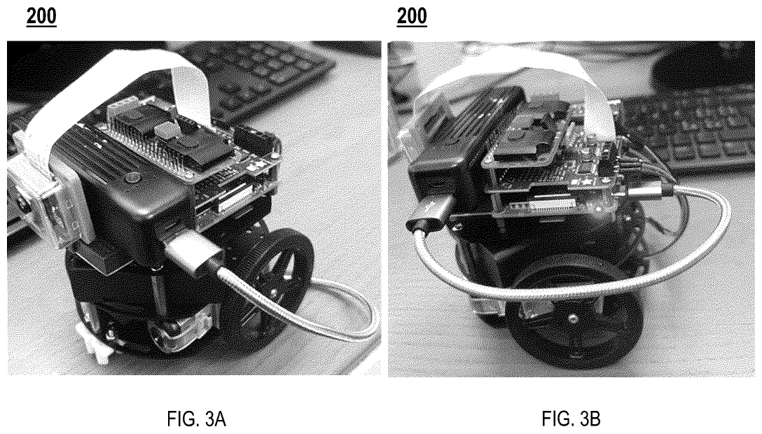

FIGS. 3A and 3B depict another exemplary embodiment of robot 200. The robot 200 may be more compact and/or include an exterior encasing, which decreases the stimulatory factors during therapy. For example, robot 200 may resemble a box shape, including all hardware enclosed within a single body, with wheels attached on each side.

In aspects, sentences may be generated using artificial intelligence and/or machine learning. For example, network 300 may be a machine learning network and/or large language model (LLM), which is designed to understand, generate, and manipulate human language. By way of example, the network 300 can simulate conversations, helping children with ASD practice language skills, turn-taking, and the rules of conversation in a low-pressure environment. This can be especially helpful for those who find real-life social interactions overwhelming. In use, the network 300 can predict and/or modify sentences generated based on feedback from the users (e.g., therapist and child). For example, network 300 can adapt their responses based on the child's communication level. For non-verbal or minimally verbal children, simple prompts or visuals could be used, while more advanced language learners might benefit from more complex conversation practice.

In aspects, network 300 can monitor a child's progress and automatically adjust a therapy program accordingly. For example, network 300 may receive as input the conversational parameters discussed above (e.g., measuring the number of turns in the conversation; the number of appropriate turn taking; the number of initiations of dialogue; the number of topics selected by the child versus followed from the conversation; the number of appropriate topics selected by the child for the given conversation; the number of appropriate words used to express a topic; the number of different words/meanings selected; and the number of verbs and pronouns appropriately used overall and mean utterance length), and output a prediction of a child's progress (e.g., needs improvement, on track, or ahead), and adjust a therapy plan accordingly.

Network 300 may connect two or more children to be able to converse with each other via their respective robot 200. In aspects, the children matched to converse may be selected based on an AI algorithm, e.g., based on the conversational parameters, to maximize a potential therapeutic benefit. For example,

The methodologies of embodiments of the present disclosure may be particularly well-suited for use in an electronic device or alternative system. Accordingly, embodiments of the present disclosure may take the form of an entirely hardware embodiment or an embodiment combining software and hardware aspects that may all generally be referred to herein as a “processor,” “circuit,” “module” or “system.” Furthermore, embodiments of the present disclosure, or portions thereof, may take the form of a computer program product embodied in one or more computer readable medium(s) having computer readable program code stored thereon.

Any combination of one or more computer-usable or computer-readable medium(s) may be utilized. The computer-usable or computer-readable medium may be a computer-readable storage medium. A computer-readable storage medium may be, for example but not limited to, an electronic, magnetic, optical, electromagnetic, infrared, or semiconductor system, apparatus, device, or any suitable combination of the foregoing. More specific examples (a non-exhaustive list) of the computer-readable storage medium would include the following: a portable computer diskette, a hard disk, a random access memory (RAM), a read-only memory (ROM), an erasable programmable read-only memory (EPROM or flash memory), an optical fiber, a portable compact disc read-only memory (CD-ROM), an optical storage device, a magnetic storage device, or any suitable combination of the foregoing. In the context of this disclosure, a computer-readable storage medium may be any tangible medium that can contain or store a program for use by or in connection with an instruction execution system, apparatus or device.

Computer program code for carrying out operations of embodiments of the present disclosure may be written in any combination of one or more programming languages, including an object oriented programming language such as Java, Smalltalk, C++ or the like, and conventional procedural programming languages, such as the “C” programming language or similar programming languages. The program code may execute entirely on the user's computer, partly on the user's computer as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Embodiments of the disclosure are referred to herein, individually and/or collectively, by the term “embodiment” merely for convenience and without intending to limit the scope of this application to any single embodiment or inventive concept if more than one is, in fact, shown. Thus, although specific embodiments have been illustrated and described herein, it should be understood that an arrangement achieving the same purpose can be substituted for the specific embodiment(s) shown; that is, this disclosure is intended to cover any and all adaptations or variations of various embodiments. Combinations of the above embodiments, and other embodiments not specifically described herein, will become apparent to those of skill in the art given the teachings herein.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. Relational terms such as “upper,” “lower,” “front” and “back,” where used, are intended to indicate relative positioning of elements or structures to each other when such elements are oriented in a particular manner, as opposed to defining an absolute position of the elements.

Certain aspects of the present disclosure may include some, all, or none of the above advantages and/or one or more other advantages readily apparent to those skilled in the art from the figures, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate aspects, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in aspects,” “in various aspects,” “in some aspects,” or “in other aspects” may each refer to one or more of the same or different example Aspects provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The aspects described with reference to the attached figures are presented only to demonstrate certain examples of the disclosure. Other elements, steps, methods, and techniques that are insubstantially different from those described above and/or in the appended claims are also intended to be within the scope of the disclosure.

What is claimed is:

1. An system for training conversation skills, dialogue interaction skills, and pragmatic language skills in a patient with autism spectrum disorder (ASD), the system comprising:
a robot having a non-humanoid non-animal-like shape;
a processor; and
a memory coupled to the processor and including instructions stored thereon, which when executed by the processor cause the system to:
record at least one of an audible or a visual expression of the patient in proximity of the robot;
record speech elicited by a therapist and the patient;
provide a predicted sentence based on the recorded at least one audible or visual expression and the recorded speech; and
audibly generate the predicted sentence, wherein the predicted sentence is pronounced by the robot in order to elicit a verbal response from and maintain a conversation with the patient.

2. The system of claim 1, wherein the at least one audible or visual expression includes at least one of an emotion, a belief, or a communicative intention.

3. The system of claim 1, wherein the at least one audible or visual expression includes at least one of a neutral, inexpressive, or normal tone.

4. The system of claim 1, wherein the recorded speech includes a pre-recorded sentence produced by a neurotypical child.

5. The system of claim 1, wherein the recorded speech includes a sentence produced by a speech synthesizer.

6. The system of claim 5, wherein the speech synthesizer includes a large language model (LLM) configured to simulate a conversation with the patient.

7. The system of claim 4, wherein the pre-recorded sentence is audibly generated by the robot using a command on a remote control device.

8. The system of claim 7, wherein the remote control device is a wireless remote control device.

9. The system of claim 7, wherein the remote control device is a wired remote control device.

10. The system of claim 1, wherein the predicted sentence is configured to initiate a piloted dialogue with the patient.

11. A computer-implemented method for conversation skills, dialogue interaction skills, and pragmatic language skills in a patient with autism spectrum disorder (ASD), the method comprising:
recording at least one of an audible or a visual expression of the patient in proximity of a robot, wherein the robot has a non-humanoid non-animal-like shape;
recording speech elicited by a therapist and the patient;
providing a predicted sentence based on the recorded at least one audible or visual expression and the recorded speech; and
audibly generating the predicted sentence, wherein the predicted sentence is pronounced by the robot in order to elicit a verbal response from and maintain a conversation with the patient.

12. The method of claim 11, wherein the at least one audible or visual expression includes at least one of an emotion, a belief, or a communicative intention.

13. The method of claim 11, wherein the at least one audible or visual expression includes at least one of a neutral, inexpressive, or normal tone.

14. The method of claim 11, wherein the recorded speech includes a pre-recorded sentence produced by a neurotypical child.

15. The method of claim 11, wherein the recorded speech includes a sentence produced by a speech synthesizer.

16. The method of claim 15, wherein the speech synthesizer includes a large language model (LLM) configured to simulate a conversation with the patient.

17. The method of claim 14, wherein the pre-recorded sentence is audibly generated by the robot using a command on a remote control device.

18. The method of claim 17, wherein the remote control device is a wireless remote control device.

19. The method of claim 17, wherein the remote control device is a wired remote control device.

20. The method of claim 11, wherein the predicted sentence is configured to initiate a piloted dialogue with the patient.
